(19) United States
(12) Patent Application Publication
(10) Pub. No.: US 2001/0049369 A1
(43) Pub. Date: Dec. 6, 2001

(54) Brimonidine compositions and methods for retinal degeneration

(76) Inventors: Monica M. Jablonski, Cordova, TN (US); Alessandro Iannaccone, Cordova, TN (US)

Correspondence Address: BARBARA S. KITCHELL, Akerman, Senterfitt & Eidson, P.A., 222 Lakeview Avenue, Fourth Floor, P.O. Box 3188, West Palm Beach, FL, 33402-3188, US

(21) Appl. No.: 09/780,210

(22) Filed: Feb. 9, 2001

Related U.S. Application Data
(60) Provisional application No. 60/181,587, filed on Feb. 10, 2000.

(51) Int. Cl. A61K 31/498

(52) U.S. Cl. 514/250

(57) ABSTRACT

The present study demonstrates that brimonidine tartrate, an alpha-2 adrenergic receptor agonist, can prevent photoreceptor cell degeneration and the associated Muller cell degenerative signs in an in vitro model of retinal degeneration and retinal detachment (separation of the neuroretina from the retinal pigment epithelium). Similar to control conditions, brimonidine allowed for the formation of highly structured photoreceptor outer segments, prevented the expression of stress markers in Müller cells and preserved the expression patterns of Muller cell markers of proper cell-cell contact and differentiation. Ultrastructural studies also indicated that brimonidine favored the formation of cell-cell junctions between photoreceptor cells and Müller cells, indicating that this phenomenon is associated with the exertion of the neuroprotective effect. The results suggest that brimonidine compounds may be utilized as an effective therapeutic agent for early and late onset retinal degenerations caused by defects in photoreceptor cells, Müller cells or both, and as an adjuvant to therapeutic success in retinal detachment surgery or macular translocation surgery for age-related macular degeneration.

This application claims priority as a continuation-in-part application based on U.S. provisional patent application Ser. No. 60/181,587, filed Feb. 10, 2000, the entire contents of which are herein incorporated by reference.

### 1.0 BACKGROUND OF THE INVENTION

1.1 Field of the Invention

The present invention concerns the use of highly selective alpha2-adrenoceptor agonists in preventing or reversing retinal degeneration. In particular, compositions and methods utilizing brimonidine are disclosed.

1.2 Description of Related Art

Brimonidine (5-bromo-6-(2-imidazolidinylideneamino)-quinoxaline) is a potent adrenoceptor agonist recognized as highly selective for the alpha2-receptor compared with the alpha1-receptor. The best documented effect of brimonidine by topical administration to the eye is a decrease in intraocular pressure (IOP), the rise of which can lead to the damage to the optic nerve head known as glaucoma. This IOP-lowering effect has been observed both in animals and in clinical trials (Burke and Schwartz, 1996; Walters 1996; Schuman, 1996; Serle et al. 1996; Wilensky 1996). Because of these properties, a topical formulation of brimonidine (brimonidine tartrate 0.2%) has been on the market for several years for the treatment of chronic open-angle glaucoma.

Osborne (1991) reported the ability of clonidine and UK 14304 which are alpha2-adrenoceptor agonists with lower selectivity for alpha2-adrenoceptors than brimonidine, to reduce cAMP levels following stimulation with forskolin and VIP (agents known to increase cAMP levels). The conclusion of the study was that in the retina, clonidine and UK 14304 inhibited cAMP activity via an inhibitory guanine nucleotide regulating protein. Osborne et al. did not evaluate the neuroprotective properties of alpha2-adrenoceptor agonists; rather, they used biochemical methods to determine the intracellular signaling pathway that is stimulated by the alpha2-adrenoceptor agonists in retinal homogenates.

Lachkar et al. (1998) and Carlsson, et al. (1999) evaluated the effect of topically applied brimonidine tartrate (a highly selective alpha2-adrenoceptor agonist) on ocular hemodynamics in human glaucoma (elevated intraocular pressure) patients. The authors demonstrated that the ability of brimonidine tartrate to reduce intraocular pressure did not affect the blood flow in the ophthalmic artery, central retinal artery, nasal artery and temporal ciliary arteries of glaucomatous patients. While these findings have important implications in the management of glaucoma, they do not address the potential use of alpha2-adrenoceptor agonists to mitigate retinal degenerations induced by genetic factors, mechanical separation (i.e., retinal detachment), inflammation, aging, or a combination of any of these.

Several studies describe the results of the administration of alpha2-adrenoceptor agonists intraperitoneally (by injection in the gut) or intramuscularly (by injection in the muscle) but virtually all these studies have attempted to evaluate the effect of alpha2-adrenoceptor agonists upon optic nerve crush or retinal ischemia and do not address the problem of treating photoreceptor cell degeneration as might be associated with retinal degenerations as they can occur by virtue of genetic factors, mechanical separation (i.e., retinal detachment), inflammation, aging, or a combination of any of these.

In addition to the aforementioned beneficial effect on intraocular pressure, Burke and Schwartz (1996) found that secondary damage following mechanical injury to a rat optic nerve was not observed when brimonidine was administered prior to the damage. Studies to establish the site of action for neuroprotective effects in rat models of optic nerve degeneration have been reported by Yoles et al (1999). In these studies, the researchers injured the rat optic nerve first by crushing it (i.e, mechanical injury), then testing several alpha2-adrenoreceptor agonists, including brimonidine, for the ability to protect against injury-induced damage. The protective effects were measured electrophysiologically by compound action potential amplitude and morphometrically by counting retrogradely labeled retinal ganglion cells. These cells are those from which the axons that form the optic nerves originate. Intraperitoneally administered brimonidine was effective in decreasing loss of retinal ganglion cells induced by the crush. These studies, of great relevance to glaucoma research, provided no indication that retinal photoreceptors or Müller cells were affected, which are the cell types predominantly affected in retinal degenerations induced by genetic factors, mechanical separation (i.e., retinal detachment), inflammation, aging, or a combination of any of these.

Later experiments were performed on an acute retinal ischemic reperfusion injury animal model. This model entails the sudden obstruction of blood flow through the ophthalmic artery, which supplies blood to the innermost layers of the retina via the retinal vessels. This circulation does not provide blood supply to photoreceptors, which are nourished via the choroid, a vascular layer present underneath the retina. These studies on the acute retinal ischemic reperfusion injury animal model showed that topically applied brimonidine was effective in decreasing ischemic retinal injury when administered 1 hour before injury, Wheeler et al. (1999). No evidence was provided that animals could be treated with brimonidine after ischemic injury to decrease the effects of ischemia. Again, Wheeler et al. examined ganglion cell survival and function, but not retinal photoreceptors and Müller cells, which are the cell types most affected in retinal degenerations induced by genetic factors, mechanical separation (i.e., retinal detachment), inflammation, aging, or a combination of any of these.

1.3 Hereditary Retinal Degenerations

To date, the only treatment that has been shown to exert a beneficial effect on one form of hereditary retinal degeneration, known as retinitis pigmentosa (henceforth referred to as RP) is dietary supplementation with 15,000 international units (I.U.) of vitamin A palmitate daily, Berson (1993). On average, this treatment slowed, but did not prevent, the progression of retinal degeneration in many patients with RP. In addition, there is recent experimental evidence suggesting that vitamin A may work for some but not all forms of RP (Li et al., 1998), and that it may possibly be harmful in certain forms of retinal degenerations in which a possible toxic accumulation of vitamin A in the retina may be taking place (such as Stargardt's disease and Leber's Congenital Amaurosis). In the latter two diseases, certain genetic changes affect the function of molecules in the retinal photoreceptors or in the retinal pigment epithelium that impair the normal metabolism of vitamin A that is necessary for normal vision to take place (Redmond et al, 1998, Weng et al., 1999).

1.4 Age-related Macular Degeneration (AMD)

AMD is the leading cause of legal blindness in the American population over the age of 60, and affects in some form almost one in three Americans over the age of 75. Also for this highly prevalent group of diseases, the therapeutic options are limited. The best available treatment consists of burning with a laser beam the vascular membranes which in some patients develop under the retina and disrupt the overlying retinal photoreceptor cells. This is the exudative (or wet) form of AMD. The treatment, however, is effective only when the disease does not affect the central region of the retina (the fovea). Moreover, it does not prevent recurrences (which take place in over 50% of the cases) and may cause damage to the retina because the burn is not localized enough to spare completely the retinal tissue. Clearly, this method of treatment does not produce very satisfactory results.

An improvement to this type of treatment has recently come from availability of compounds that bind to lipids selectively expressed in these abnormal vascular membranes and that can be photo-activated by gentle warming with a weak laser beam. This treatment is called photo-dynamic therapy (PDT), and accomplishes the ablation of the vascular membranes from within, thanks to the pooling of the photosensitive dye inside them (and not in the normal surrounding vessels). While PDT shows promise in the management of wet AMD, this treatment too does not protect photoreceptors from disease damage. In fact, multiple re-treatments are necessary (up to 5-6 within the first two years from the first PDT application).

Two surgical approaches are currently being evaluated: (1) submacular surgery to remove the neovascular membranes and (2) retinal translocation that causes a transient retinal detachment and simultaneous rotation of the retina in order to position a healthier portion of the retinal tissue in the foveal area (where sharp vision takes place).

There are several limitations to these surgical approaches. These include: (1) damage to the retinal pigment epithelium (RPE) caused by subretinal membrane “stripping” and (2) damage to the photoreceptor outer segments caused by induced retinal detachment in the translocation procedure.

Other treatments are being investigated, such as anti-angiogenic compounds that can inhibit the formation of these harmful neovascular membranes underneath the retina, and will likely have a key impact on the management of wet AMD in the future. These too, though, are targeted at the management of the complicating factor, and not at protecting the overlying photoreceptors from degenerating. Therefore, none of these treatments would rescue the photoreceptors, and therefore restore vision, unless combined with agents capable of protecting the photoreceptors themselves.

For the most common form of AMD, the atrophic (or dry) form, which accounts for more than 80% of AMD, there is no treatment whatsoever, nor any proven means to prevent its formation, and photoreceptor degeneration in the macula ensues in this disease. As with RP, to which this disease is much more intimately connected than previously believed, there is need for effective prevention and treatment.

1.5 Deficiencies in the Prior Art

Retinal degeneration is becoming of increasing interest and concern as the population becomes older and age-related deterioration of major organs overtakes disease as a medical issue. Retinal detachments can be repaired surgically from an anatomical standpoint, but often the functional outcome is not completely satisfactory. Steps aimed at improving the viability of a detached retina prior to and after surgery may improve significantly the functional outcomes. Genetic mutations occurring in retinal photoreceptors, pigment epithelium or Müller cells often lead to degenerative changes in the retina of affected patients of all ages. To date, there is a dearth of means for stopping or slowing down effectively these degenerative processes. Therefore, there is a clear need for treatments that will slow or even reverse these conditions.

### 2.0 SUMMARY OF THE INVENTION

The present invention addresses several of the problems encountered in attepts to develop therapies and treatments for degenerative conditions of the retina, and is particularly focused in providing compositions designed to be used topically in treating conditions involving retinal degeneration.

The present invention employs selective alpha2-adrenoceptor agonists, including brimonidine tartrate and reformulated drug derivatives of brimonidine or other selective alpha2-adrenoceptor agonists, to treat retinal degeneration. The disclosed compositions are considered to be particularly useful in patients afflicted with the following clinical conditions: (1) retinal detachment; (2) post-retinal translocation surgery due to choroidal neovascularization associated with exudative age-related macular degeneration; (3) genetic mutations of retinal photoreceptors that induce degenerative changes in the retina; (4) genetic mutations of retinal pigment epithelium that induce degenerative changes in the retina; and, (5) genetic mutations of Müller cells that induce degenerative changes in the retina.

Highly selective alpha2-adrenoceptor agonists may be used as a therapy for experimental and human retinal degenerations induced by physical separation from the retinal pigment epithelium or genetic factors. Previously reported degenerative conditions have been induced by mechanical injury, ischemia, or similar insults. The results disclosed here are based on use of selective alpha2-adrenoceptor agonists, exemplified by brimonidine, to promote the formation of organized retinal structures that appear to be similar to normal retinas with adherent retinal pigment epithelium (RPE).

The alpha2-adrenoceptor agonists employed in the disclosed compositions are highly selective for alpha2 subtype adrenoceptors. Published studies by various workers have evaluated the efficacy of other alpha adrenoceptor agonists with high affinities for the alpha2-adrenoceptor but with lower selectivities for alpha2 with respect to alpha1-adrenoceptors. Such compounds, for example clonidine has varying pharmacodynamic profiles.

The disclosed methods employ selective alpha2-adrenoceptor agonists, including brimonidine or reformulated derivatives, delivered topically in the eye. This delivery route is expected to increase patient compliance because of the ease-of-use affiliated with topical means such as instilling a drop in the eye and the low incidence of side effects documented for brimonidine tartrate.

Formulations of brimonidine and related alpha2-adrenoceptor agonists are advantageously employed as ophthalmic solutions. Such opthalmic solutions are of particular interest, for example, in the treatment of detached retinas such as arising from genetic mutations or as a consequence of macular degeneration. Thus for treatment of individuals with this condition, an amount of a brimonidine composition would be administered to the eye of a subject in need of treatment in the form of an opthalmic preparation prepared in accordance with conventional pharmaceutical practice, see for example “Remington's Pharmaceutical Sciences”, 15^{th} edition, pages 1488-1501 (Mack Publishing Company, Easton, Pa.).

The ophthalmic preparation will contain a brimonidine compound or a pharmaceutically acceptable salt thereof in a concentration from about 0.01 to about 1% by weight, preferably from about 0.05 to about 0.5% in a pharmaceutically acceptable solution, suspension or ointment. Some variation in concentration will necessarily occur, depending on the particular compound employed, the condition of the subject to be treated and the like, and the person responsible for treatment will determine the most suitable concentration for the individual subject. The ophthalmic preparation will preferably be in the form of a sterile aqueous solution containing, if desired, additional ingredients, for example preservatives, buffers, tonicity agents, antioxidants and stabilizers, nonionic wetting or clarifying agents, viscosity-increasing agents and the like.

Suitable preservatives for use in such a solution include benzalkonium chloride, benzethonium chloride, chlorobutanol, thimerosal and the like. Suitable buffers include boric acid, sodium and potassium bicarbonate, sodium and potassium borates, sodium and potassium carbonate, sodium acetate, sodium biphosphate and the like, in amounts sufficient to maintain the pH at between about pH 6 and pH 8, and preferably, between about pH 7 and pH 7.5. Suitable tonicity agents are dextran 40, dextran 70, dextrose, glycerin, potassium chloride, propylene glycol, sodium chloride, and the like, such that the sodium chloride equivalent of the ophthalmic solution is in the range 0.9 plus or minus 0.2%.

Suitable antioxidants and stabilizers include sodium bisulfite, sodium metabisulfite, sodium thiosulfite, thiourea and the like. Suitable wetting and clarifying agents include polysorbate 80, polysorbate 20, poloxamer 282 and tyloxapol. Suitable viscosity-increasing agents include dextran 40, dextran 70, gelatin, glycerin, hydroxyethylcellulose, hydroxmethylpropylcellulose, lanolin, methylcellulose, petrolatum, polyethylene glycol, polyvinyl alcohol, polyvinylpyrrolidone, carboxymethylcellulose and the like. The ophthalmic preparation will be administered topically to the eye of the subject in need of treatment by conventional methods, for example in the form of drops or by bathing the eye in the ophthalmic solution.

### 3.0 BRIEF DESCRIPTION OF THE DRAWINGS

The drawings form part of the present specification and are included to further demonstrate certain aspects of the present invention. The invention may be better understood by reference to one or more of the following drawings in combination with the detailed description of specific embodiments presented herein:

FIG. 1 Illustrates the microanatomy of the retina.

FIG. 2 Illustrates the process for analysis of morphology; process for immunocytochemistry of Müller cells markers: glial fibrillary acidic protein (GFAP), glutamine synthetase and N-cadherin.

FIG. 3A Light level micrograph illustrates that in the presence of the RPE, nascent photoreceptor outer segments are composed of stacked flattened membranous saccules arranged in an orderly array (arrows).

FIG. 3B Electron micrograph illustrates that in the presence of the RPE, organized rod outer segments are comprised of individual discs of similar diameter and are completely enveloped by a plasma membrane. In addition, calycal processes emerge from the inner segment and surround the proximal portion of the outer segment (arrows).

FIG. 4A Light level micrograph illustrates that when placed in culture without a closely apposed RPE, intact retinas continue to produce large amounts of outer segment membrane. However, the membranes are arranged as large whorls with little evidence of normal disc stacking. Most of the membrane profiles do not appear to be associated with any particular photoreceptor, and form a dense mat at the outer retinal surface.

FIG. 4B Electron micrograph illustrates that when cultured in the absence of a closely apposed RPE, nascent outer segment membranes are elaborated in a multitude of lengths that form a very “jagged” silhouette or whorl-like profile. The outer segments are surrounded by a plasma membrane. Calycal processes are not present.

FIG. 5A Light level micrograph illustrates that the addition of 0.0001% brimonidine permits nascent outer segments to organize into stacked well-organized individual segments, with a morphology very similar to control retinas in which outer segments were elaborated in the presence of the RPE. (Compare FIGS. 3A, 4A, 5A.)

FIGS. 5B and 5C Electron micrograph illustrates that the addition of 0.0001% brimonidine to retinas that are maintained in the absence of the RPE permitted nascent outer segments to form highly organized structure, very similar to retinas with an adherent RPE. In addition, calycal processes are found immediately adjacent to the outer segment and many other “processes” are surrounding the organized outer segments (arrows).

FIG. 6 Electron micrograph illustrates that in the presence of brimonidine, Müller cell endfeet hypertrophy and form a bridge between adjacent photoreceptors in the area of the outer limiting membrane (OLM)(arrow). Adherens junctions are seen on either side of the hypertrophied area.

FIG. 7A Glutamine synthetase, a Müller cell enzyme with neuroprotective properties, is heavily expressed throughout the entire cytoplasm of the Müller cells. Label (arrows) indicates the presence of the antigen in retinas with an adherent RPE.

FIG. 7B Removal of the RPE prevents the expression of glutamine synthetase by Müller cells.

FIG. 7C Brimonidine allows Müller cells to express glutamine synthetase in the absence of the RPE (arrows).

FIG. 8A GFAP, a marker of Müller cell injury, is not expressed in control retinas with an adherent RPE.

FIG. 8B GFAP is, however, upregulated in Müller cells of retinas that have been cultured in the absence of the RPE (label seen as dark spots, arrows). Also, there is significant cell loss in the inner layers of the retina.

FIG. 8C Brimonidine prevented the upregulation of GFAP expression in retinas in which the RPE has been removed indicating that brimonidine may be stabilizing Müller cells as evidenced by the lack of GFAP expression.

FIG. 9 Illustrates the amount of outer segment membranous material. The addition of 0.0001% brimonidine to the culture medium stimulated a marked increase in the amount of opsin in the RPE-deprived retinas compared to both retinas cultured without brimonidine and also to control eyes maintained with an intact RPE.

### 4.0 DESCRIPTION OF ILLUSTRATIVE EMBODIMENTS

There is an unmet need for improved treatments for this blinding group of retinal diseases. The present work illustrates the application of brimonidine compounds to this clinically and genetically heterogeneous group of diseases. Brimonidine compounds may also be useful in those diseases where photoreceptors degenerate even though they do not themselves harbor genetic defects that are present in neighboring cells, namely the Müller cells.

To date, at least one disease is known to be caused by a Müller cell defect, X-linked juvenile retinoschisis (XLJR). This condition causes progressive retinal splitting (schisis) which leads to retinal detachments and ultimately also photoreceptor damage. These patients are expected to be effectively treated with brimonidine.

More particularly, it is believed that the disclosed innovative treatment will exert a significant beneficial effect on patients with early dry AMD; prevent the formation of dry AMD; and be useful as an adjuvant to the surgical approaches to wet AMD by compensating for the RPE damage involuntarily (and unavoidably) caused by the membrane stripping and by promoting preservation of healthier photoreceptors to the translocation procedures.

4.1 Retinal Photoreceptor Neuron

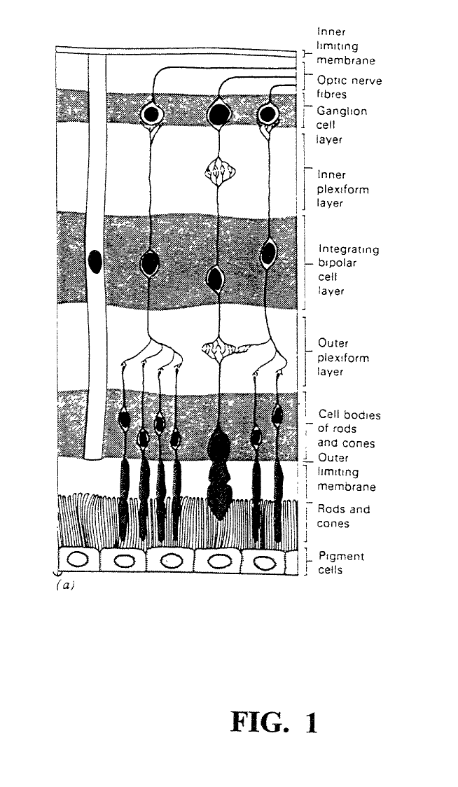

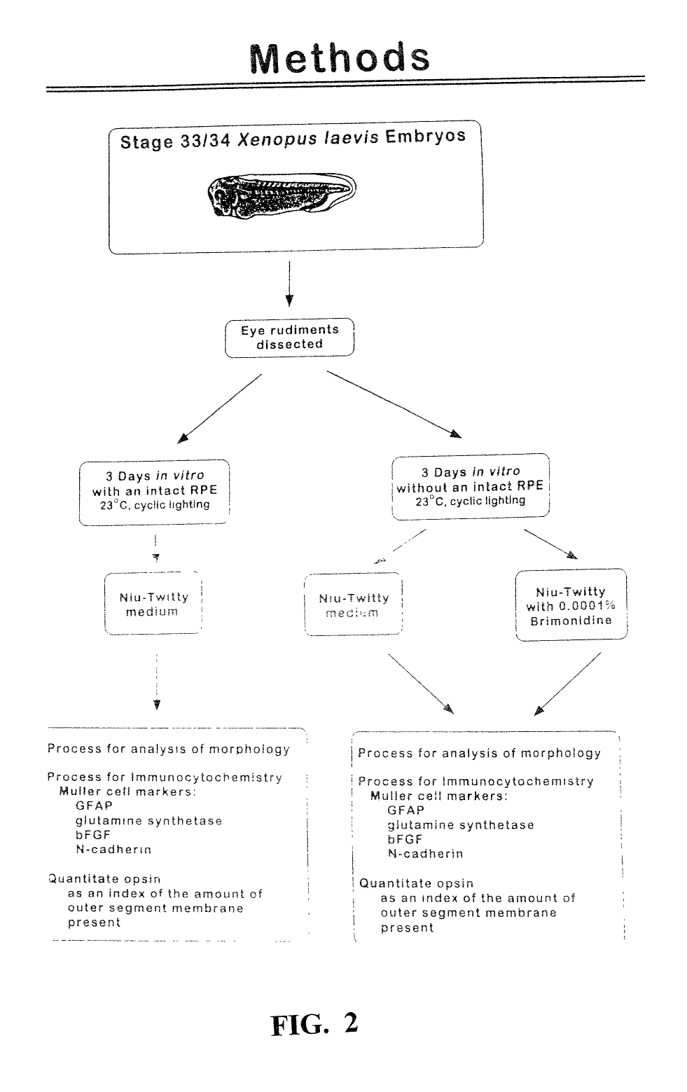

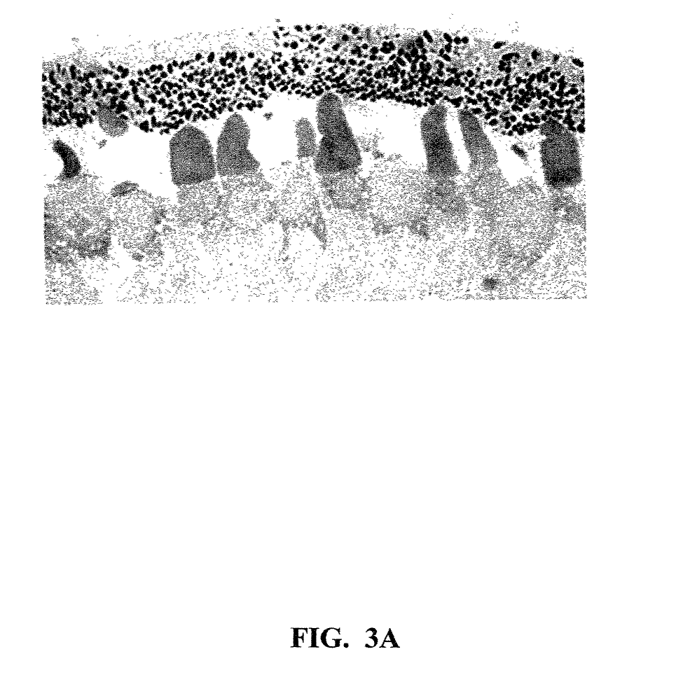

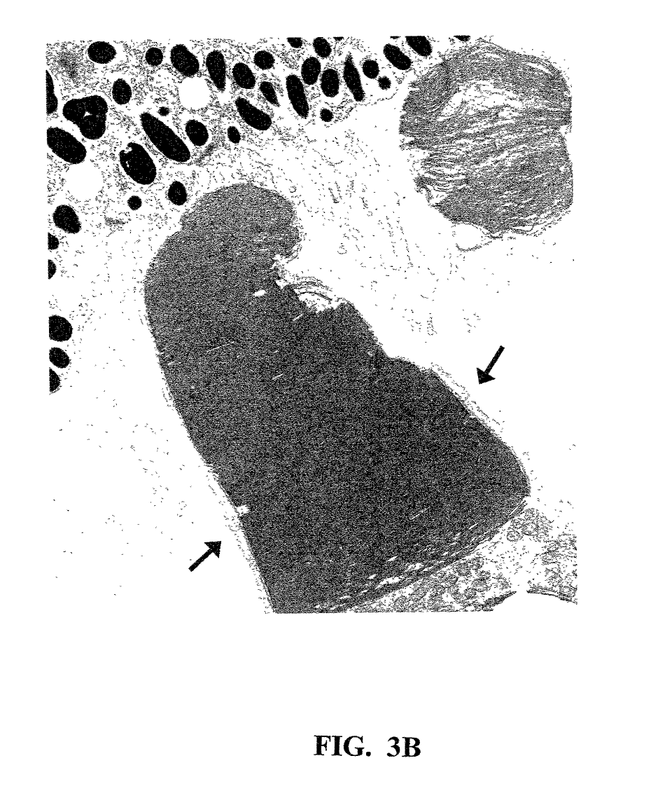

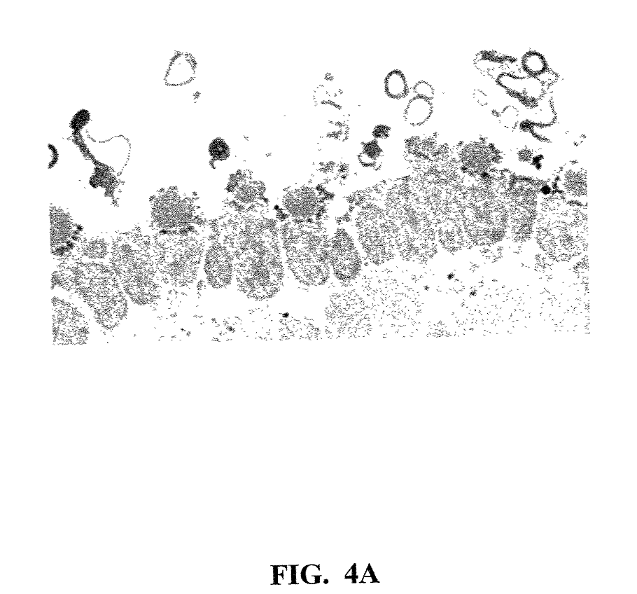

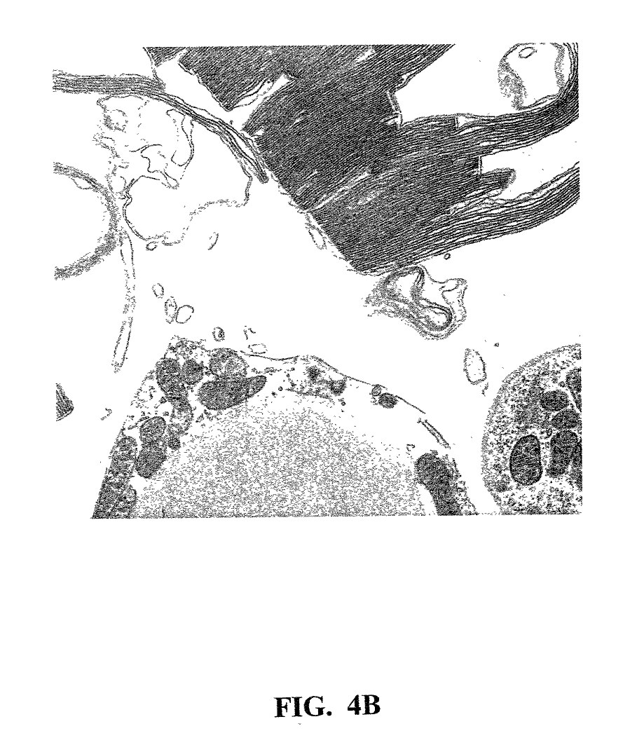

The eye develops as an out-pouching of the diencephalon of the brain. The retina, which is comprised of several distinct neural subtypes along with radial glia (i.e. Müller cells), lines the inside of the eye and it is here that the first basic steps of vision take place. One type of retinal neuron, the photoreceptor, is responsible for capturing photons of light and transducing that light into chemical signals. The array of signals originating from retinal photoreceptors is ultimately relayed to the brain where images are processed and perceived as “sight”. Photoreceptors are highly polarized and ultrastructurally unique cells (FIG. 1). At one pole of the neuron is the highly ordered outer segment, while at the other pole is the chemical synapse. The outer segment is the photosensitive portion of the photoreceptors, in which the phototransduction cascade takes place. This chain of photochemically-induced events blocks the influx of cations into the photoreceptor cell. The resultant hyperpolarization rapidly propagates to the synaptic terminal, inducing a release of the neurotransmittor glutamate toward the second-order neuron of the visual pathway, the bipolar cell.

The outer segment of the photoreceptor is composed of an array of stacked membranous saccules in perfect register (FIG. 1). Due to the huge metabolic workload to which the outer segment components are continually exposed, the outer segment membranes are continuously renewed. Newly synthesized membranous addition occurs at the proximal end of the outer segment, which is connected to the inner segment via the connecting cilium. Concurrent with membrane addition, shedding of the outer segment takes place at the distal tip, where the outer segments are phagocytized by the adjacent RPE. It is well established that disorganization of the outer segment is associated with degeneration of that same photoreceptor cell and loss of sight. Indeed, a disruption of photoreceptor outer segment is common to both human and animal models of various types of retinal degenerations. Although at a superficial evaluation, this scenario appears to be quite intuitive and fairly straightforward, the exact mechanism(s) that lead to photoreceptor degeneration are not yet completely understood.

4.2 Microanatomy of the Retina

The retina is made up of 10 thin layers (FIG. 1). The first steps of vision take place in rods and cones, collectively termed retinal photoreceptors. These neurons are located at the outermost aspect of the retina and are connected to bipolar cells (second station of the visual neural pathway) which, in turn, are connected to ganglion cells (third station). The axons of the ganglion cells form the optic nerve fiber layer, which is the innermost layer of the retina. The fibers converge into the scleral canal to form the optic nerve, which connects the eye to the brain. The optic nerve head is the only part of the nerve that is visible inside the eye. With a 90° turn, the fibers enter the canal and exit the eye, travel behind the eye inside the orbit and reach the next station of the visual pathway, situated in the middle of the brain, the lateral geniculate body. From there, the neural impulses are sent to the visual cortex in the brain, where the sensory event of vision takes place as we customarily experience it.

4.3 Mechanisms of Retinal Damage

There is a tremendous difference in the physiology and pathophysiology of the different subpopulations of retinal cells, and especially between ganglion cells and photoreceptors.

Diseases that affect photoreceptors do not harm retinal ganglion cells, and vice-versa. Biochemical pathways that are active in one cell population do not take place in the other and vice-versa. The two layers receive completely independent vascular supplies. The ganglion cells are nourished by the retinal vessels, that originate from the retinal artery which penetrates the eye through the optic nerve and branches off in smaller vessels that coat the innermost layers of the retina, embedded in the ganglion cell and optic nerve fiber layer. These vessels play no role in the support of photoreceptors, which are nourished by the underlying choroid by controlled diffusion, regulated by the Bruch's membrane and the retinal pigment epithelium (RPE).

4.3.1 Optic Nerve Crush

Optic nerve crush studies, as the term well explains, consists of damaging the optic nerve in the portion exposed behind the eyeball by means of crushing. Since retinal ganglion cells are modified brain neurons dislocated in the retina in order to connect it to the remainder of the brain, they also experience a well-known phenomenon, that of retrograde degeneration. In other words, the crushing of the axons at a site remote to the originating cells (located inside the eye) sends retrograde signals that cause the death of the mother cell. No such phenomenon applies to retinal photoreceptors, cells that have no counterpart in the rest of the human body. Retrograde degeneration of retinal ganglion cells does not affect by any means retinal photoreceptors.

Elegant experiments of optic nerve sectioning by Niemeyer and coworkers have clearly shown that a flash electroretinogram (ERG), that originates from photoreceptors, bipolar cells and Müller cells, is unaffected by complete optic nerve sectioning, even though no visual message reaches the brain and the animal is de facto blind. Therefore, efficacy of compounds that prevent or reduce the damaging effects on ganglion cells induced by crushing has no relation to the expectation of protecting photoreceptor cells from degeneration.

4.3.2 Retinal Ischemia

Retinal ischemia studies are induced by ligation of the retinal artery behind the eye. The interrupted blood supply induces marked ischemic damage of the inner retina, but not to the outer retina where photoreceptors are located. The most affected cells are the ganglion cells. In the clinical setting, this becomes evident as development of optic disc pallor, a well-known maker of the secondary effect of the ischemia. The pallor is expression of atrophy, and indicates presence of permanent damage of the optic nerve fibers, that are the axons of the ganglion cells. At times, also the bipolar cells can be affected by ischemia, because they are situated at the watershed area between the retinal and the choroidal vascularization. The impairment of bipolar cells due to ischemia can be appreciated through the ERG, in which the bipolar cell-mediated response (known as the b-wave) can be truncated if the ischemia is sufficiently severe. Brimonidine has been shown to partially prevent this b-wave truncation due to ischemia. Ischemia has no effect on the a-wave, which is the ERG component contributed by photoreceptors.

4.3.3 Photo-induced Retinal Damage

Light-induced retinal damage targets photoreceptors. Light-induced retinal damage is a well-characterized phenomenon in rodents and in albino animals. Rodents are highly susceptible to light damage, because their visual system is made to function in total darkness. In effect, rodents are not daytime animals, and their retinas are very sensitive to light (more so than the human retina) as well as to the ultraviolet portion of the spectrum of visible light, to which the human retina is not sensitive at all. The pupillary response of rodents to a beam of light is particularly telling: very small amounts of light, which in humans elicit minimal pupil constriction, evoke marked pupil contraction in mice (Pennesi IOVS, 1998). Therefore, exposure of these animals to inordinate amount of bright light distresses their visual system far more than the human one. The same applies to albino animals, in which the light-absorbing pigment granules present in the retinal and ciliary pigment epithelium are absent, causing severe glare and inordinate exposure of the retinal tissue to bright lights. In either case, and more so in the latter, rodents and other animals are susceptible to severe photoreceptor light damage.

There are a number of animal models for human retinal degenerations, and their similarity to the human ones in striking. The differences, however, are also significant. One of the most important ones is the fact that marked light exposure exacerbates considerably the genetically-determined degeneration in these animal models.

The effect of light in exacerbating genetically related retinal degeneration does not take place in the human eye. Although repeatedly invoked in a number of conditions, no conclusive evidence exists that light directly causes retinal damage in humans. If such a mechanism exists in the human eye, it is suspected that it may act chronically as an oxidative insult, which in turn may predispose to other toxic side effects from free radicals. The possible involvement of light damage in retinitis pigmentosa (henceforth referred to as RP), the prototype of progressive genetic retinal degenerations has been explored over 30 years ago in a controlled experiment by Berson. His study consisted in patching one eye of a group of patients with RP for an extended period of several years. The fellow eye was used as a control. At the end of the follow-up, the progression of RP had been the same in each eye, showing that light damage had no effect on the rate of progression of the disease.

Albinism is also a common disease of humans. There is no evidence that exposure to light causes retinal deterioration in these patients, despite the severe glare that they experience as a side effect of the lack of the light-absorbing dark pigment.

Age-related macular degeneration (henceforth erred to as AMD) is yet another important and highly prevalent human disease, there is little evidence to suggest that light damage causes a significant role in predisposing to the disease. In the Chesapeake Bay epidemiological study, there was no evidence that fishermen (inherently exposed to higher amounts of sunlight than the average population) had a greater incidence of AMD.

4.4 Mechanisms of Photoreceptor Damage

4.4.1 Apoptosis

Not all means of cell death are equal. Cell death occurs through two main phenomena, those accompanied by reaction and inflammation, termed necrosis, and the quieter ones that kill the cell from within via the activation of existing programmed cell death pathways, termed apoptosis (from the Greek, “to die on itself”). Apoptosis is a widespread phenomenon that occurs at all times in the human body. At birth, a number of unnecessary cells born in excess of the needs of each organ are “terminated” via activation of apoptotic pathways. This is a physiologic (that is, normal) response. The same happens with aging. In the disease state, premature and inappropriate apoptotic events take place. Apoptosis has been shown to exist in both retinal light damage and genetically-determined retinal degenerations. Interestingly, however, the avenues (that is, the apoptotic pathways) that can lead to cell death are numerous and not interrelated.

Apoptosis arising from light damage is c-fos- and c-jun-mediated. The c-fos and c-jun pathways are activated when an extracellular agent injures the cell from the outside. Not surprisingly, then, this pathway is involved in light damage, because the agent is administered from the outside of the cell. There is no evidence that these pathways are activated in genetically-determined retinal degenerations, in which the insult leading to cell death is coming from within the cell. To test the hypothesis that inhibition of the c-fos/c-jun pathway protects animals from retinal degeneration, genetically engineered animals have been constructed which do not express these genes (so-called knock-outs). Again not surprisingly, these animals did not experience any significant decrease in the rate of genetically-determined retinal degeneration, demonstrating that apoptotic pathways active in light damage have little bearing on the outcome of genetically-determined retinal degenerations.

4.4.2 Photoreceptor Degeneration Resulting from Removal of the RPE

RPE removal in vitro has previously been shown to result in dysmorphogenesis of the outer segment and photoreceptor degeneration in the embryonic Xenopus laevis eye rudiment. The present study has demonstrated that the addition of brimonidine, a highly selective and water soluble α₂-adrenergic agonist with reported neuroprotective actions, (Burke and Schwartz (1996); Yoles et al. (1999)) reverses this degenerative pattern in an in vitro model.

Alpha-2 (α2-) adrenergic receptors have been identified in the retina. The α2 subtype is the main α receptor in the retina (Bittiger et al. (1980); Osborne et al. (1982)). It has been localized primarily to the inner plexiform layer, ganglion cell layer and photoreceptor layer in both the human retina and in several other animal species (Zarbin et al., 1986).

4.4.3 Photoreceptor Degeneration Resulting from Mutations in RPE Genes

The possibility that, as in the Royal College of Surgeons (RCS) rat, a defect in the RPE may be the cause of retinal degenerations in humans has long been suspected. In recent years, support for this assumption has come from the identification of disease-causing mutations in RPE-expressed genes in several forms of retinopathies. For example, mutations cosegregating with disease manifestations have been found in the RPE65 gene (Marlhens, 1997; Gu, 1997) and in the cellular retinaldehyde binding protein (CRALBP) gene (Maw, 1997). RPE65 mutations are responsible for early-onset forms of retinitis pigmentosa (RP) and Leber's congenital amaurosis, both inherited in an autosomal recessive fashion (Marlhens, 1997; Gu, 1997).

The product of the RPE65 gene is critical to the metabolic pathways that transform vitamin A taken up from the choroidal circulation into 11-cis-retinal, which is required for photoreceptor phototransduction (Redmond, 1999). It is believed that null RPE65 mutations deplete photoreceptors of this indispensable metabolite and cause an abnormal intracellular accumulation of an 11-cis-retinal precursor, all-trans retinyl ester, that leads to RPE cell death and is detrimental to photoreceptor survival (Redmond, 1999). Severe forms of autosomal recessive retinitis pigmentosa (RP) are also linked to CRALBP mutations (Maw, 1997). CRALBP is downstream in the same metabolic pathway as RPE65, and is likely to cause RP via a two-fold mechanism similar to that described above.

4.4.4 Age-related Macular Degeneration

RPE dysfunction and/or cell loss is also causative for photoreceptor damage in age-related macular degeneration (AMD). AMD is a clinically heterogeneous condition, the precise genetic causes and/or predisposing factors are not fully understood (Seddon, 1998; Klein, 1999). AMD is the leading cause of blindness in people over 65 years of age. In the USA, the population in this age range was 30 million in 1995, and it is expected to rise to 70 million by the year 2050. Up to 20% of people older than 65 may develop AMD and this number increases to 37% by age 75. It is obvious from these numbers that this condition is reaching epidemic proportions and will have a highly significant social and financial impact on society. The clinical presentation of AMD presents typically as one of two distinct forms, either as dry (atrophic) or the wet (exudative) (reviewed in [Lewis, 1992]).

In brief, the atrophic form of AMD correlates with less severe visual loss; however, it is more prevalent. It presents with RPE clumping, as well as geographic and non-geographic RPE atrophy, often producing a relative scotoma in central vision. Exudative AMD affects fewer individuals than the dry form; however, it affects more severely the visual function of patients. It often presents with choroidal neovascular membranes, RPE detachment, and formation of a disciform scar, with resultant photoreceptor degeneration (reviewed in Lewis, 1992). Surgical excision of the neovascular membrane removes the fibrotic membrane from under the retina, but often this procedure removes areas of RPE. It has also been reported that choroicapillaris atrophy can follow CNV removal, thus exacerbating the photoreceptor damage (Nasir, 1997). In both forms of this potentially debilitating condition, the RPE either remains in its normal position next to the retina but is suffers from atrophic changes, or the RPE is stripped from its adherent position next to the photoreceptors. In either case photoreceptors are deprived of the supportive role of the RPE. It is therefore evident that the visual loss suffered by those elderly patients affected by AMD is primarily, although perhaps not exclusively, due to RPE dysfunction or loss.

From this brief synopsis, it is evident that problems with the genetic composition, physical presence or function of the RPE leads to degeneration of the underlying retina. However, the underlying cellular mechanism(s) is not yet known. Herein we postulate a potential mechanism for the degeneration and further suggest that this may be a mechanism through which neuroprotective factors prevent retinal demise.

4.5 Treatments for Retinal Degeneration

4.5.1 Effect of α₂-Adrenergic Agonists on Photoreceptors

Systemic administration of xylazine and clonidine, two α₂-adrenergic receptor agonists, has been shown to protect the photoreceptors of albino rats from light damage by upregulating bFGF expression in photoreceptors (Wen et al., 1996). The mechanisms of action were shown to be (1) α₂-receptor-mediated, because pretreatment with yohimbine, an α₂-adrenergic antagonist, prevented the protective effect; and, (2) independent of a reduction in systemic blood pressure induced by xylazine and clonidine (i.e., the main clinically known effect of α₂-adrenergic agonists). These findings suggested a direct effect of xylazine and clonidine on photoreceptor α₂-adrenergic receptors.

4.5.2 Surgical Intervention for Retinal Detachment

Therapy for retinal detachment is surgical. By and large, treatment of this condition (in most cases, traumatic in origin) is anatomically successful, provided that the detachment has not been present for too long a period of time. Regarding restoration of function after the surgery, however, the outcome is more variable.

The results shown by the invention suggest that treatment of eyes with retinal detachment prior and after surgery will stimulate the maintenance of healthier photoreceptor outer segments and promote an overall more resilient retinal tissue, thereby enhancing the satisfactory functional outcomes of anatomically successful surgeries for retinal detachment.

Alpha-2 adrenergic agonists have been known for many years, and many have long been available on the market, first for oral administration as anti-hypertensive agents, and subsequently as anti-glaucoma agents by topical ocular application, owing to their ability to reduce intraocular pressure.

The most common of the systemically administered alpha-2 adrenergic agonists is clonidine, from which also brimonidine was derived. Alpha-2 adrenergic agonists have a number of side effects that make them a second choice in the management of hypertension. The most important are neuro-mediated effects, since these agonists elicit their action via a “central” mechanism, that is by modulating alpha-2 adrenergic receptors at the cerebral level more so than peripherally (as is done by the other anti-hypertensive agents). Drowsiness and fatigue are two major and frequent side effects of alpha-2 adrenergic agonists, as well as dryness, dangerous interactions with other neuroactive agents, precipitation of depressive attacks, and renal side effects. While none of these side effects is totally eliminated by topical administration, their frequency is drastically reduced, making this category of drugs far more appealing.

Brimonidine is not the only alpha-2 adrenergic agonist available for topical administration. Apraclonidine (Iopidine, Alcon) is a “relatively selective” alpha-2 adrenergic agonist that is currently used to lower intraocular pressure, especially following certain laser surgeries associated with ocular hypertensive spikes after the treatment. In its present formulation, the drug is not well tolerated for long term use due to a number of local side effects, such as ocular redness, dryness, discomfort, allergy, itching and burning, up to drug-induced conjunctivitis. In addition, there is currently no evidence that apraclonidine could exert the same neuroprotective effects that we have so far documented, nor has it been tested in any of the experimental conditions tested by others (see previous art).

It is expected that apraclonidine, and perhaps clonidine itself which is available in Europe as an eye drop to lower intraocular pressure, and has been shown to be useful in the light damage model via intraperitoneal injections, is likely to exert a neuroprotective effect similar to the effect with brimonidine. However, due to their far lower selectivity for alpha-2 adrenergic receptors than brimonidine which appears to account for the majority of the observed effects, higher concentrations of clonidine and apraclonidine would be required to attain the same effect, increasing the already ample array of undesired side effects noted for these compounds. In a snowball effect, this would diminish patients' compliance reducing both applicability and ultimate therapeutic success. Thus, while these compounds may attain an effect similar to that of brimonidine, they would represent a second choice agents due to the anticipated need for higher dosages and/or more frequent installations.

Should these drugs be reformulated in such a way to reduce significantly their side effects and/or more importantly to increase their potency in terms of alpha-2 adrenergic receptor agonism, they would be good candidates for a proposed therapeutic initiative.

4.6 Müller Cells

Muller cells are essential for photoreceptor homeostasis. Müller cells, the radial glial cells of the retina, ensheath the photoreceptors from the synaptic terminus to the level of the photoreceptor inner segment, forming a very close physical relationship. It has been demonstrated that Müller cells offer metabolic and trophic support to photoreceptors which promotes their survival (Reichenbach, 1993; Newman, 1996; and Cao, 1997). This is especially true for rod photoreceptor cells, which originate from the same precursor as Müller cells. Rods, as well as other neurons, form retinal columnar structures which appear to be very heavily dependent on metabolic support from Müller cells, more so than ‘extracolumnar’ retinal neurons such as cones (Reichenbach, 1993). In addition, Müller cells express voltage-gated ion channels, neurotransmitter receptors and various uptake carrier systems which enable them to modulate the activity of retinal neurons by regulating the extracellular concentration of neuroactive substances (Reichenbach, 1997). Previous studies have shown that Müller cells support photoreceptors, in particular, by buffering the local microenvironment from excess extracellular potassium and glutamate that accumulates as a result of the photoransduction cascade and neurotransmitter release at the synaptic terminus, respectively (Ripps, 1985; Germen, 1997). The glutamate released at the photoreceptor synapse is internalized by the Müller cells by means of high-affinity carrier systems and converted to the non-toxic amino acid glutamine by glutamine synthetase. This enzyme is widely represented throughout the entire body of the Müller cells.

4.6.1 Role for Müller Cells

MCs are known to exert an important role in maintaining retinal integrity and function both during and after retinal development (Newman and Reichenbach, 1996). In a rodent model of retinal injury (Wen et al., 1995) showed that MCs exert an important protective action also on photoreceptors (Wen et al., 1995). An upregulation of bFGF in these injured retinas was documented. Proper MC function requires normal cell-cell interaction (Moscona and coworkers, 1998). Maintenance of proper cell-cell interaction appears to be a key factor in promoting and sustaining the protective role of MCs on photoreceptors (see also Wohabrebbi et al., and Jablonski et al.).

Two lines of evidence connect the α₂-adrenergic agonist-mediated effect on photoreceptors to MCs. First, systemically administered xylazine and clonidine activate selectively and specifically mitogen-activated protein kinase (MAPK) phosphorylation in MCs (Peng et al., 1998); and second, increased bFGF in the culture media further upregulates bFGF expression in MCs (Cao et al., 1997). These observations suggest a possible virtuous cycle promoted by the α₂-adrenergic agonist action on the retina, possibly via a dual action on both the photoreceptors and MCs.

The inventors have demonstrated that the α₂-agonist, brimonidine, exerts a protective effect on photoreceptors in an in vitro model, allowing for highly structured outer segment formation similar to control retinas. This effect was observed at the morphological (LM and EM), and immunohistochemical level, as well as when quantifying the amount opsin (slot blot analysis). This protective action appears to be mediated by a direct effect on photoreceptors, an indirect activation of MCs, or both.

The morphological (EM) and immunohistochemical (n-cadherin) evidence of MC stimulation, OLM enhancement and promotion of tighter junctions between MCs and photoreceptors induced by the addition of brimonidine to the culture media suggests a favorable effect of this α₂-adrenergic agonist on cell-cell interactions between MCs and photoreceptors.

Yoles et al. (1999) have documented neuroprotective effects of brimonidine in ganglion cells using a rat model. The present work shows a remarkable effect on photoreceptor cells, thus for the first time indicating that brimonidine may be an effective therapeutic agent for certain forms of retinal degenerations or other conditions where disruption of RPE integrity may lead to permanent loss of photoreceptor function.

In other applications, it has been shown that the described in vitro model provides a practical screening method for identifying compositions with potential therapeutic applications for treating retinal degenerations.

4.6.2 Müller Cells “React” to RPE Dysfunction

While it has been known for many years that an intact RPE-neural retina complex is a requisite for survival of photoreceptors and therefore visual function, the precise mechanism(s) through which this occurs is not fully understood. In conditions in which the RPE carries a genetic mutation or is removed physically, both photoreceptors and Müller cells undergo degenerative or reactive changes. In animal models, induced separation of the neural retina from the juxtaposed RPE layer results in very rapid cone and rod outer segment degeneration (Guérin, 1993) with the degree of recovery of cell morphology and function being negatively correlated with the duration of the detachment (Guérin, 1989; Guérin, 1993); and Müller cells upregulate expression of GFAP (Erickson, 1987; Eisenfeld, 1984), vimentin and tubulin (Lewis, 1995, Okada, 1990).

In the RCS rat, it has been demonstrated that a defect in the RPE results in photoreceptor degeneration unless growth factors are injected into the subretinal space (Faktorovich, 1990) or an RPE transplant is performed (Li, 1991). Müller cells are also affected in this model of retinal degeneration, with an elevated expression of GFAP and apical process hypertrophy into the subretinal space (Roque, 1990). Under areas of transplanted RPE, however, less GFAP immunoreactivity is detected and apical process sprouting is inhibited (Li, 1993).

4.6.3 Müller Cell Dysfunction in Patients

Several histopathologic reports on human donor retinas with retinal degenerations have been published (Green, 1985; Rodrigues, 1987; Farber, 1987; Flannery, 1989; Birnbach, 1994; Li, 1994; Birnbach, 1994; Li, 1995; Li, 1995; Santos, 1997; Milam, 1998; and Green, 1999). Because most retinal degenerations are due to primary defects of the photoreceptors or of the RPE, reports have focused primarily on these two cell types. The notion that Müller cells are also abnormal in these retinas has long been known (Rodrigues, 1987; Farber, 1987). However, in most reports Müller cell abnormalities have been mainly described as ‘reactive gliosis’, i.e. viewed as a secondary expression of cellular stress and essentially a repair phenomenon aimed at ‘filing in the space’ left vacant by dying photoreceptors and other retinal neurons.

To the best of our knowledge, we are unaware of histopathologic studies on donor tissues of patients with retinal degenerations that have focused on characterizing the pattern of abnormalities of Müller cells. In fact, this may be difficult in human donor tissues, which become typically available only in the late disease stages which may confound the scenario and preclude the investigation of this aspect.

Clinical evidence, however, indicates that extra-photoreceptoral abnormalities do exist in patients with RP. A study by Cideciyan et al. (Cideciyan, 1993) specifically demonstrated a disproportionate reduction of the b-wave of the electroretinogram (ERG) in patients with RP, indicating that other factors in excess of mere photoreceptor damage were present in those patients. While this study (Cideciyan, 1993) could not identify the cellular localization of the abnormality, bipolar cell abnormalities in donor RP retinas have not been yet documented, while abnormalities of Müller cells are well known. It is therefore conceivable that the patients evaluated by Cideciyan et al. had a greater than average Müller cell dysfunction which became evident in the ERG.

Several recent studies utilizing a modified ERG technique which allows for a better dissection of the retinal cell contributions to the various components of the ERG response suggest that this may well be the case (Falsini, 1994; Falsini, 1999). In fact, Falsini and co-workers (Falsini, 1999) provided evidence of a selective Müller cell dysfunction in the central retina of RP patients with well preserved visual acuity. This study provides evidence that Müller cell abnormalities are neither an end-stage nor a passive, secondary phenomenon in RP, but that a selective and measurable Müller cell dysfunction does exist at stages when photoreceptor function is still well preserved, at least in the central region of the retina.

The minimum subcellular characteristics that define a healthy retina are now known. While ample evidence demonstrates that RPE cell loss or dysfunction results in photoreceptor degeneration and Müller cell reaction (Eisenfeld, 1984; Erickson, 1987; Faktorovich, 1990; Roque, 1990; Li, 1991; Li, 1993, Guérin, 1993; Stiemke, 1984; Jablonski, 1999), the correlation between these events has not been fully delineated.

The present work reveals key differences in the ultrastructural and protein expression characteristics of healthy and brimonidine-protected retinas compared to dysmorphic retinas. Healthy retinas possess highly organized photoreceptor outer segments, photoreceptor calycal processes, and adherens junctions between all adjacent photoreceptors and Müller cells.

Under control conditions in which the RPE was present during in vitro morphogenesis, it is now demonstrated that GFAP protein expression is undetectable, as would be expected in healthy retinas. In these same healthy retinas, glutamine synthetase immunopositive labeling in a radial pattern was detected, following the path of Müller cells through the entire retinal thickness from the outer to inner limiting membranes. Glutamine synthetase immunolabeling was heavy in the plexiform layers where synaptic communication occurs between neurons and Müller cells remove extracellular glutamate, converting it to glutamine via catalysis with glutamine synthetase. It therefore follows that a high concentration of glutamine synthetase is localized in these regions, where it protects retinal neurons by removing potentially harmful glutamate from the extracellular spaces, as suggested by Gorovits.

Glutamine synthetase, localized exclusively in Müller cells, is also a key enzyme in glial-neuronal neurotransmitter recycling and a potent neuroprotectant. Previous evidence also indicates that the expression of glutamine synthetase is indicative of cell-cell contact between Müller cells and retinal neurons. The data disclosed herein regarding the ultrastructural presence of the adherens junction along with N-cadherin, an extracellular key component of the adherens junction, provides further evidence for the role of the junction in supporting protein expression patterns in Müller cells.

Each of the above outlined ultrastructural findings and protein expression patterns is lacking in degenerating retinas. The reported results indicate that RPE-deprived retinas undergo degeneration demonstrated by dysmorphogenesis of outer segment membranes, loss of adherens junctions and calycal processes, alterations in the expression patterns of Müller cell-specific proteins, and some cell loss within the inner and outer nuclear layers. In these same retinas, GFAP was upregulated, similar to that documented in various forms of retinal injury induced by retinal detachment, light damage, a genetic defect in the RPE (i.e. the RCS rat), and age-related macular degeneration. Glutamine synthetase was not detected by immunocytochemical localization in the Müller cells of degenerating retinas and N-cadherin expression was not detectable.

The inventors have demonstrated that brimonidine tartrate is able to mitigate the degenerative alterations induced by RPE removal in both photoreceptors and Müller cells by permitting the proper folding and organization of nascent outer segment membranes, allowing for the formation of calycal processes of photoreceptors, promoting the formation of adherens junctions, preserving the expression and localization patterns of GFAP, glutamine synthetase and N-cadherin. The subcellular cytoarchitecture of the outer retina and the protein expression patterns therein are very similar to retinas that were maintained in the presence of an intact RPE. The most striking features are: the organization of the outer segment, which is critical to photoreceptor survival; the formation of the adherens junction, which may stimulate both cell types to promote proper protein expression patterns; and stabilization of glutamine synthetase expression, a potent neuroprotectant.

The ultrastructural presence of the adherens junctions exclusively in healthy and brimonidine-protected retinas as evidenced with EM microscopy in conjunction with the expression of N-cadherin and immunolocalization of glutamine synthetase ties the physical presence of the junctions with the expression of this key Muller cell enzyme. These data, in conjunction with previous studies, suggest that cellular interactions between photoreceptors and Müller cells are required for supporting the proper ultrastructure of both cell types. This cell-cell contact in turn may allow for the expression of photoreceptor and Müller cell-specific proteins that potentially play a role in maintaining retinal structural integrity and health (Jablonski, 1999). Brimonidine allows for the formation of the adherens junction and promotes the formation of proper subcellular cytoarchitecture that may have downstream effects on outer segment membrane assembly and stability. In the absence of the correct ultrastructure, photoreceptor and Muller cell malformation follows. These results may have particular relevance when evaluating the protective effect of neuroprotective agents upon retinal degenerations of various causes.

4.7 Results

4.7.1 Light Microscopy (LM) Morphological Findings

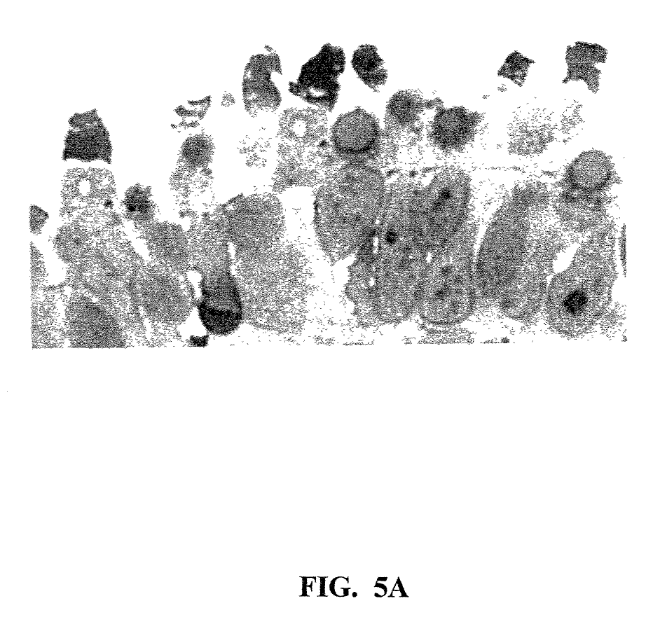

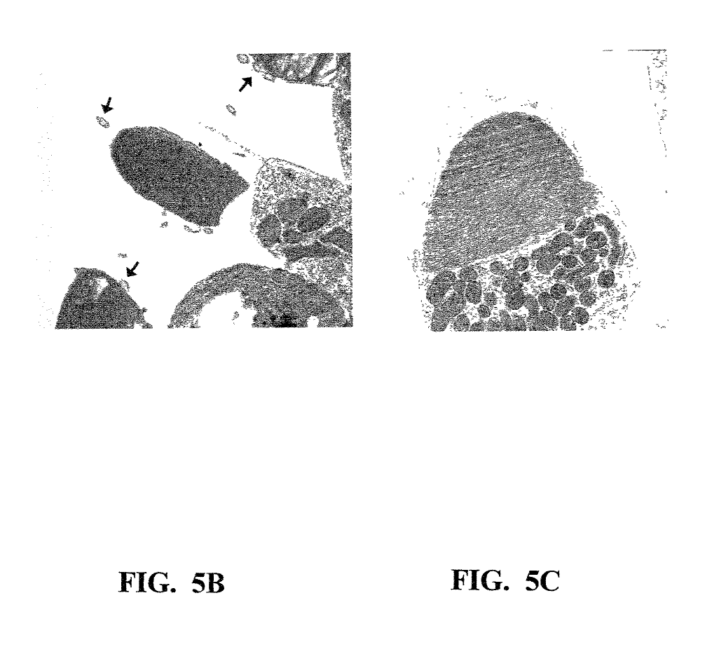

The addition of 0.0001% brimonidine to the culture media in RPE-free eyes induced a highly structured organization of the photoreceptor outer segment membranes, similar to control retinas in which the RPE was present (FIG. 5A).

In the presence of brimonidine, the Müller cells (MC) showed prominent nuclei with darkly stained radial processes that are continuous with the outer limiting membrane (OLM). Compared to MCs cultured without the addition of brimonidine, the processes were significantly more prominent, indicating a hypertrophic response of MCs (FIG. 5A).

4.7.2 Electron Microscopy (EM) Morphological

The better preservation of the outer segments in the in vitro model of photoreceptor degeneration in the presence of 0.0001% brimonidine is more noticeable at the EM level. Significantly better preservation of outer segment disc morphology and organization is observed, compared to eyes cultured without the RPE (FIG. 5B).

Calycal processes around photoreceptor outer segments are seen in brimonidine-treated eye rudiments, but were not observed in untreated RPE-free eyes (FIG. 5C).

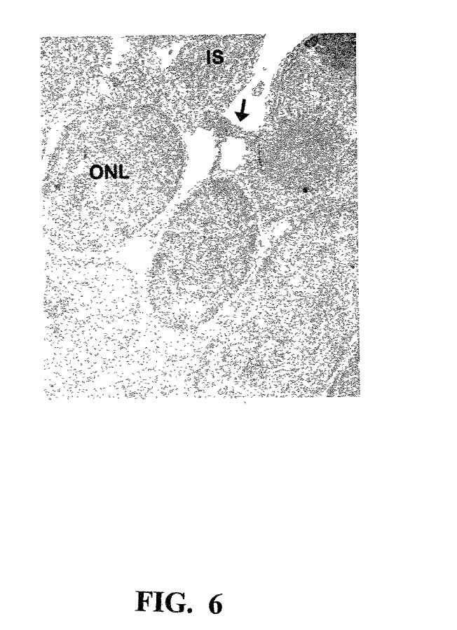

The darkly stained MC processes and thickened OLM noted on LM in the brimonidine-treated eyes can be readily visualized at the EM level: T-shaped MC hypertrophic processes in the end-foot region form close junctions (zonulae adherentes) with neighboring photoreceptor inner segments. This was observed neither in RPE-free eyes nor in control retinas, suggesting that the OLM hypertrophy reflects a specific effect on MCs mediated by brimonidine (FIG. 6).

4.7.3 Light Microscopy Immunocytochemistry

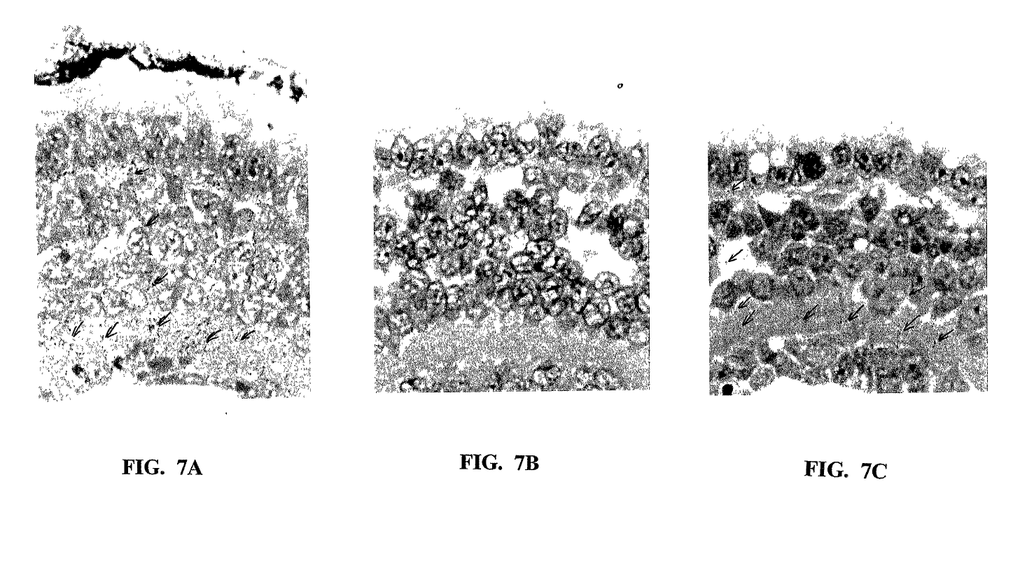

Glutamine synthetase (GS) immunoreactivity was well preserved in brimonidine-treated eye rudiments, (FIG. 7C) and was similar to the pattern of expression observed under control conditions (FIG. 7A).

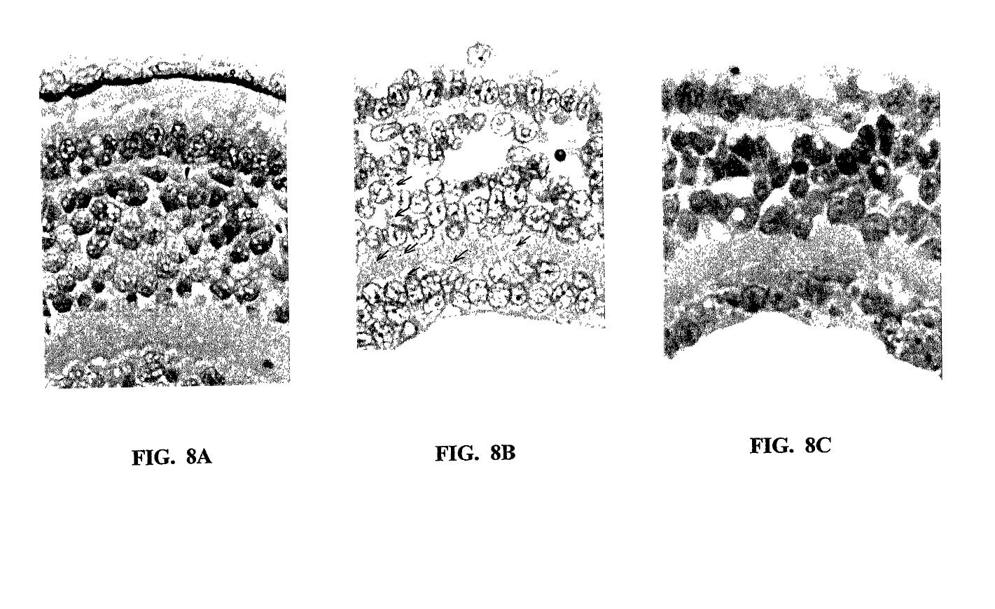

The upregulation of GFAP induced by RPE removal (FIG. 8B) was virtually abolished by the addition of brimonidine to the culture media (FIG. 8C).

Consistent with the MC hypertrophy and enhanced formation of zonulae adherentes at the OLM level, n-cadherin immunolocalization patterns (severely disrupted by RPE removal) was preserved following addition of 0.0001% brimonidine. (Data not shown.)

4.7.4 Slot Blot Analysis

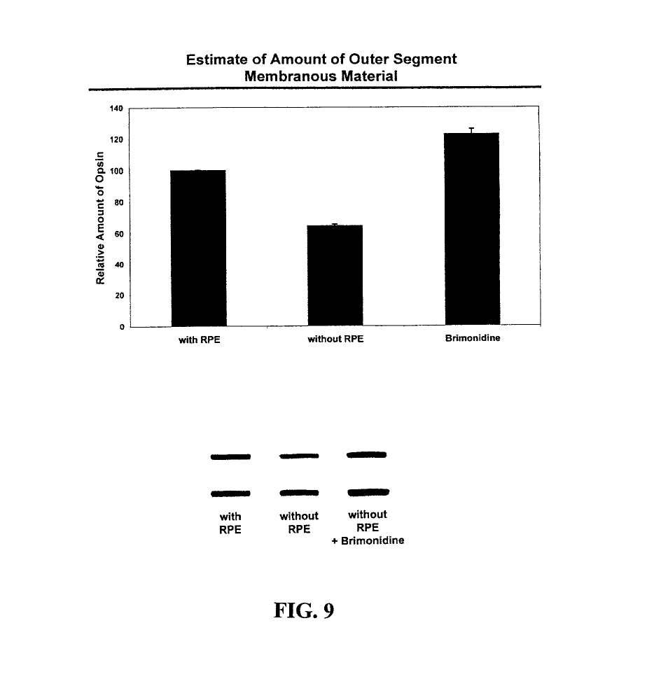

The addition of 0.0001% brimonidine to the culture media resulted in a marked increase in the amount of opsin/eye in RPE-free eyes compared to eyes cultured without brimonidine; opsin concentration was also elevated also compared to control eyes (FIG. 9).

### 5.0 EXAMPLES

The following examples are included to demonstrate preferred embodiments of the invention. It should be appreciated by those of skill in the art that the techniques disclosed in the examples which follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### 5.1 Example 1

Regeneration of Retinal Morphology

The following studies were performed on Xenopus embryos. Briefly, eye rudiments were removed from stage 33/34 Xenopus embryos. The RPE was peeled from the outer retinal surface and eyes were cultured for 3 days in Niu-Twitty (NT) medium. The same experiment was performed in NT medium containing 0.001% brimonidine. Control conditions included eye rudiments cultured with the RPE and eyes allowed to mature in vivo to an equivalent developmental stage. Retinal morphology was assessed, carefully evaluating PRs and MCs.

5.1.1 Culture of Developing Retinas

The culture preparation used in these studies has been previously described (Hollyfield, 1974; Lolley, 1977; Stiemke, 1994; Stiemke, 1995; and Jablonski, 1999). The handling of animals was in accordance with the Declaration of Helsinki and The Guiding Principles in the Care and Use of Animals (DHEW Publication, NIH 80-23). Human chorionic gonadotropin (Sigma Chemical Co., St. Louis, Mo.) was to induce adult Xenopus laevis to breed. The external staging system of Nieuwkoop and Faber was used to determine retinal maturity (Nieuwkoop, 1956). Embryos and isolated eyes were maintained under cyclic lighting conditions (12 hour light: 12 hour dark) in all experiments, rudimentary eyes were removed from embryos at state 33/34, just as photoreceptor outer segments are beginning to form (Stiemke, 1994). Eyes were maintained in vitro for three days at 23° C. in Niu-Twitty medium (Jacobson, 1967) after which they were fixed for subsequent analyses.

Using this culture protocol (removal at state 33/34 and maintenance at 23° C. for three days), isolated retina-RPE complexes have reached approximately state 42 of the in vivo developmental scale, characterized by complete stratification of the retina and fully mature photoreceptors complete with well developed outer segments and synaptic connections (Hollyfield, 1979; Stiemke, 1994). When appropriate, the overlying RPE was gently peeled away from the neuroepithelium using finely polished forceps, leaving the underlying retina exposed to the culture medium. Eye rudiments without a closely adherent RPE were cultured in Niu-Twitty media alone, Niu-Twitty containing 0.001% brimonidine. Eyes allowed to mature in vitro in the presence of an adherent RPE in Niu-Twitty medium alone were used as controls.

5.1.2 Ultrastructural Morphological Assessment

After three days of in vitro development, eyes were grossly examined under a dissecting microscope for integrity and smoothness of the neuroepithelial or RPE surface to ensure that all eye rudiments were intact. Any eye with an uneven surface or which had many loose cells associated with it was discarded. For structural analysis, eyes were fixed in Tucker fixative (2% glutaraldehyde and 1% osmium tetroxide in 0.05M phosphate buffer), dehydrated, and embedded in Araldite/EMbed812 (Electron Microscopy Sciences, Fort Washington, Pa.). Because Xenopus retinas mature most rapidly in the posterior pole, ultrastructural analysis was performed on thin sections taken exclusively from this area so as to ensure that cells with equivalent stages of maturation would be compared. Careful attention was also paid to the orientation of the eyes so that the planes of section were respected.

Thin sections were cut in the vertical and horizontal planes, collected on 200 mesh copper grids and viewed on a JEOL 2000 electron microscope. To verify ultrastructural findings on horizontal sections, photoreceptor cells with intervening Müller cells were evaluated for the presence of adherens junctions between adjacent photoreceptors and Müller cells and Müller cell apical processes. For this analysis, eight adjacent photoreceptor-Müller cell complexes of two eyes from three separate experiments were examined.

5.1.3. Preparation of Retinas for Immunocytochemical Analysis and Ultrastructural Assessment

After three days in vitro at 23° C., the eyes reached approximately stage 42 of the in vivo developmental scale at which time retinas were fully stratified with elongated outer segments and retinal cells have normal protein expression patterns (Hollyfield, 1979; Steimke, 1994; Jablonski, 1999 and Jablonski, 1999). Prior to fixation, eyes were grossly examined under a dissecting microscope for integrity and smoothness of the RPE surface. Any eye with an uneven surface or which had many loose cells associated with it was discarded. Four to five eyes from three separate experiments were evaluated immunocytochemically and ultrastructurally.

For immunocytochemical localization analysis, eyes were fixed in Davidson fixative (32% ethanol, 2% formalin, 11% acetic acid), dehydrated, and embedded in Unicryl (Electron Microscopy Sciences, Fort Washington, Pa.). One micron thick sections taken through the posterior pole of the eye were cut and collected on microscope slides. Sections were incubated in 5% goat serum (Vector Laboratories, Burlingame, Calif.) in phosphate buffered saline (PBS), rinsed in PBS and incubated overnight in primary antibody. The following antibodies were used: anti-GFAP, 1:100 dilution (A2D4 [Dahl, 1985]; anti-GS, 1:1000 (Chemicon International, Inc., Temecula, Calif.); and anti-N-cadherin, 1:100 dilution (Zymed, Inc., San Francisco, Calif.). Gold-conjugated goat anti-mouse, anti-rabbit or anti-rat secondary antibodies, as appropriate, were applied to the tissue sections (1:50 dilution for two hours, ultrasmall gold particle size) followed by silver enhancement, as described by the manufacturer (Electron Microscopy Services, Fort Washington, Pa.). Controls included absence of primary antibody.

Retinal sections were viewed on a Nikon Eclipse E400 microscope equipped with Sensys Color Camera (Photometrics, Tuscon, Ariz.) and images were collected using MetaMorph Imaging System software (Universal Imaging Corporation, West Chester, Pa.). Two images were collected of each retinal section: a brightfield image which shows the morphology of the tissue; and another image taken with epipolarized light which shows only the immunolabeling pattern. The epipolarized image was color enhanced (red) and merged with the brightfield image so that the specific immunolabeling patterns could be easily distinguished. Because tissues were not post-fixed in osmium, photoreceptor outer segment structure is not readily visible in retinas processed for immunocytochemical analysis.

For structural analysis, eyes were fixed in Tucker fixative (2% glutaraldehyde and 1ˆ osmium tetroxide), dehydrated, and embedded in Araldite/EMbed812 (Electron Microscopy Sciences, Fort Washington, Pa.). Thin sections were cut in the vertical plane through the posterior pole of the eye, collected on 200 mesh copper grids and viewed on a JEOL 2000 electron microscope.

5.1.4. Opsin Quantitation

Four sets of nine to ten eyes were collected, ground and solubilized with sodium cholate detergent (Sigma Chemical Colo., St. Louis, Mo.). Extracted proteins were applied in duplicate to Hybond-P membrane (Amersham Pharmacia Biotech, Buckinghamshire, England) using a slot blot apparatus (Biorad, Hercules, Calif.). Blots were blocked in blotto (5% in PBS) for one hour followed by incubation in primary antibody overnight at 4° C. (anti-opsin; B630N [Röhlich, 1989] at 1:10,000 dilution). The ECF Western blotting kit (Amersham Pharmacia Biotech, Buckinghamshire, England) was used according to manufacturer's protocols. Blots were scanned on a Storm 860 image analysis system (Molecular Dynamics, Sunnyvale, Calif.) and data were quantified using ImageQuant software version 1.1 (Molecular Dynamics, Sunydale, Calif.). For each of the repetitions, data were normalized to values obtained for retinas maintained in the absence of Müller cell inhibitor. The student's T-test was employed to determine differences between eyes that were maintained with an intact RPE and any other culture condition.

5.1.5 Results

In control retinas maintained with an intact RPE, nascent PR outer segments were composed of stacked flattened membranous saccules arranged in an orderly arrray. When cultured without the RPE, PRs elaborate extensive amounts of outer segment membrane, arranged as large whorls with little evidence of normal disc stacking. MC radial processes were increased in number and appeared more tortuous, filling in gaps between degenerating retinal cells. Eye rudiments cultured with 0.0001% brimonidine showed a highly structured organization of the outer segment membranes, similar to retinas in which the RPE was present. MCs have prominent nuclei with darkly stained radial processes that are continuous with the external limiting membrane.

### 5.2 Example 2

In vivo Application

Preliminary studies will be conducted in normal animals comparable to those to be studied for the in vivo model experiments in order to determine the intravitreal concentrations of brimonidine reached via topical administration. These concentrations, at the dosages utilized clinically for other applications (2% solution of brimonidine tartrate) on a twice daily regimen have been used for rabbit and primate models [450 and 100 nanomolar (nM), respectively]. Small concentrations [<15 nM in the rabbit] can reach the fellow eye via the systemic circulation. The intraocular concentration is likely to be inversely proportional to the surface area available for absorption. Therefore, the mouse eye (a convenient model) is expected to reach concentrations approximately 10 times higher than the primate eye (considered by analogy comparable to the human eye). Once verified, the dosage of brimonidine in eyedrops will be adjusted accordingly (10:1 dilution, i.e. 0.02%) to mimic more closely the intraocular concentrations that would be reached during human application.

An animal model that is accepted as comparable to human disease, either naturally occurring (e.g. rds mouse, rd mouse, Abyssinian cat, Briard dog) or genetically engineered (so-called transgenic animals, carrying mutations in target genes identical to those that cause human disease, or knock-outs, lacking the expression of a given gene to mimic a human equivalent caused by null mutations in that same gene) will then be selected. The existing models are so numerous that they pose the embarrassment of choice. Ideally, we will select at least two animal strains, one with a slow-and one with a fast-occurring retinal degeneration, in order to determine whether the intervention is effective in both scenarios. Animals will be maintained under tightly-controlled environmental illumination levels, to minimize the risk that light damage mechanisms may be activated in the studied models.

The experiments will be conducted in a double-blind controlled randomized fashion by administering drops in each eye of the animal being treated. One eye at random will receive brimonidine, the fellow eye will receive a placebo solution containing only the vehicle in which brimonidine tartrate is dissolved. The eye drops will be administered via bottles labeled R for the right eye and L for the left eye.

Those conducting the study will be blinded as to the content of each bottle until completion of the experiment. A second set of untreated animals will also be followed as further control at the same time points. The need for an untreated control is to determine the in vivo concentrations sufficient to exert a protective action in retinal degenerations. Therefore, a means of excluding the possibility that small concentrations of drug reaching the fellow placebo-treated eye via the systemic circulation are not exerting a protective effect that could hamper the interocular comparison and to the incorrect conclusion that the drug is ineffective is required.

A mouse model will be analyzed. Mice are the most diffuse and best characterized models, and pose significantly small costs than larger animals such as cats, dogs and pigs. Even though the latter more closely resemble the human eye with a cone-rich area in lieu of the human macula (such region is absent in rodents, cats and dogs), the mouse model is one generally acceptable as applicable to retinal degeneration in humans. The costs associated with larger animal maintenance, however, need to be justified by prior evidence of an in vivo efficacy. The inherent difficulties in administering an eye drop in a pig eye twice daily in the awakened state are also a significant deterrent in considering this animal model as a suitable choice.

### 5.3 Example 3

In Vivo Retinal Degeneration

This example illustrates the advantages of the route of administration, which is preferably topical in the form of eye drops. This provides several advantages, both at the preclinical level and for a potential clinical treatment trial.

The ease of administration, as opposed to intramuscular, intravenous, intraperitoneal, and subcutaneous routes, is immediately apparent. This is advantageous also over the oral (per os) administration, which also may have lower compliance. Most importantly, the topical route would allow the use of far smaller doses of the drug that would be required per any systemic route. This, in addition to the prevailing absorption of the drug through the cornea, minimizes the systemic side effects of the drug of choice.

Another significant advantage of this route applies to the proposed treatment of genetically determined retinal degenerations. These diseases affect both eyes, typically in a symmetric fashion. The causes of retinal degenerations are diverse. The rate of progression of the diseases is different for each and one would expect the response to treatment also to be different. This is true in both animals and humans, creating the need for control subjects (this being either a strain of animal or a patient treated with placebo).

A limitation of this mandatory choice is that no two living organisms are created equal and each is exposed to distinct environmental variables, the modulatory effect of which on genetic diseases is largely unknown. Therefore, a control will never be a perfect match to the treated subject. All these limitations must be considered in evaluating many of the reports utilizing animal and human studies.

The possibility of using a topically applicable drug offers the unique opportunity to overcome this critical limitation by utilizing the fellow eye of the subject as an internal control. In so doing, the perfect matched control to the treatment arm is found, allowing unsurpassed control for any genetic and epigenetic (that is, environment and the like) confounding factor.

5.3.1 Pre-clinical Application

First, preliminary studies will be conducted in normal animals comparable to those studied for the in vivo experiment to determine the intravitreal concentrations of brimonidine reached via topical administration. These concentrations, at the dosages utilized clinically (2% solution of brimonidine tartrate) twice daily are already known for the rabbit and for primates [450 and 100 nanomolar (nM), respectively]. Small concentrations [<15 nM in the rabbit] can reach the fellow eye via the systemic circulation.

The intraocular concentration is likely to be inversely proportional to the surface area available for absorption. Therefore, the mouse eye is expected to reach concentrations approximately 10 times higher than the primate eye, considered by analogy comparable to the human eye. Once verified, the dosage of brimonidine in the eyedrops would be adjusted accordingly (predicted to be a 10:1 dilution, i.e., 0.02%) to mimic more closely the intraocular concentrations that would be reached during human application. It is anticipated that administration of the eye drops between twice (every 12 hours=b.i.d.) and four times (every 6 hours=q.i.d.) daily will be sufficient to maintain therapeutic levels throughout the day. The b.i.d. regimen is the currently recommended one for brimonidine tartrate to lower intraocular pressure, but this may be insufficient to maintain constant therapeutic levels during the day at the retinal level.

We will then select an animal model that is comparable to human disease, either naturally occurring (e.g., rds mouse, rd mouse, Abyssinian cat, prcd dog) or genetically engineered (so-called transgenic animals, carrying mutations in target genes identical to those that cause human disease, or knock-outs, lacking the expression of a given gene to mimic a human equivalent caused by null mutations in that same gene). The existing models are numerous. Ideally, at least two animal strains will be selected, one with a slow- and one with a fast-occurring retinal degeneration, in order to determine whether the proposed intervention is effective in both scenarios. Animals will be maintained under tightly-controlled low environmental illumination levels, to minimize the risk that light damage mechanisms may be activated in the studied models.

The experiments will be conducted in a double-blind controlled randomized paired design by administering drops in each eye of the animal being treated. One eye at random will receive brimonidine tartrate solution, whereas the fellow eye will receive a placebo solution containing only the vehicle in which brimonidine tartrate is dissolved. The eye drops will be administered via bottles labeled R for the right eye and L for the left eye.

The researchers will be blinded as to the content of each bottle until completion of the experiment.

A second set of untreated animals will also be followed as further control at the same time points. The need for an untreated control is that the in vivo concentrations sufficient to exert our postulated protective action in retinal degenerations are unknown. Therefore, we need a means of excluding with certainty that the small concentrations of drug reaching the fellow placebo-treated eye via the systemic circulation were not exerting a protective effect that could hamper the interocular comparison and lead us to conclude incorrectly that the drug(s), or combinations/formulations thereof, were ineffective.

The first step will be to analyze a mouse model. Mice are the most diffuse, most accepted and best characterized models, and pose significantly smaller costs than larger animals such as cats, dogs and pigs. The latter would be ideal, because their eyes more closely resemble the human eye with a cone-rich area in lieu of the human macula (such region is absent in rodents, cats and dogs). The costs associated with their maintenance, however, need to be justified by prior evidence of in vivo efficacy. The inherent difficulties in administering an eye drop in a pig eye twice daily in the awakened state are also a significant deterrent in considering this animal model as a suitable first choice.

The assessment of the efficacy will be both at the functional and at the histological level. Protection of function will be assessed by means of signals recorded from the eye in vivo with contact lenses or other electrodes which will collect the responses from the eye surface. This method, called the electroretinogram (ERG), is used routinely in clinical practice and is the gold standard of retinal function assessment. The amplitude of the signals originating from each eye will be the main functional outcome measure. Protection of the structure of the retinal tissue will be analyzed under light and electron microscopy, observing and grading tissue morphology and staining it with markers of cell integrity such as, but not limited to, those utilized in the preliminary investigations.

### 5.4 Example 4

Human in vivo Tests

Human application will follow essentially the same strategy as above. There will be two main differences compared to the preclinical studies. An obvious one is that the outcome measures will be only functional and not histological, due to the impossibility of obtaining samples of retinal tissue from the enrolled patients. Functional measures will not be limited to the ERG, because a number of so-called “psychophysical” parameters can be measured in human beings. These parameters rely on the active response of the patient. Examples are visual acuity, visual field size and sensitivity light (which is tested with automated static perimetry and dark adaptation).

A second important difference is that ethical considerations make it mandatory that, when a “standard treatment” exists, it be administered to all patients enrolled in the trial and that the efficacy of the new treatment be compared to the standard one. We have already addressed the deficiencies and limitations of the previous art in this regard. With this caveat in mind, 15,000 I.U. of vitamin A palmitate per os (orally) will be administered to all patients. The eye drops will be administered in a double-blinded randomized fashion, identical to what done in the preclinical studies.

The efficacy of the alpha-2 adrenergic agonist brimonidine tartrate will be assessed between the two eyes, comparing the eye treated with vitamin A systemically and placebo (vehicle) topically vs. the eye receiving both vitamin A systemically and brimonidine topically.

While comparison vs. another treatment modality may make it more difficult to ascertain small therapeutic effects, ethical issues preclude an alternative choice. In addition, by “guaranteeing” the possible benefit of vitamin A to all patients, compliance and enrollment should be maximized.

There is no scientific reason to predict that vitamin A and brimonidine will antagonize each other's effects, thereby making it likely that the two treatments will be synergistic, at least in those patients responsive to both. Finally, preliminary findings suggest that the effect of brimonidine will be sufficiently large to disclose sizable interocular differences at the end of the follow-up period (not less than three years).

This length of time is required as untreated RP has been shown to experience consistent and statistically significant progression in all patients only after three years.

5.5 Example 5

Alternative Formulations and Methods of Administration

5.5.1 Slow-release Compositions

The dosage and frequency of administration will be decided subsequently to the experience derived from the preclinical studies. It is likely that a three time per day (t.i.d.) frequency may be required to maintain retinal therapeutic levels. Should this be the case, compliance may become an issue.

However, reformulations of the current brimonidine tartrate eye drop may be pursued to circumvent this problem; for example, a compound similar to the GELRITE™ gellan gum (a registered trademark of Merck & Co., Inc.) could be added to the current formulation. GELRITE™ is a purified anionic heteropolysaccharide derived from gellan gum. An aqueous solution of GELRITE™, in the presence of a cation, has the ability to gel. This enhances the permanence of the drug in conjunctival sac, transforming it into an analog of a depot formulation, that is a long-acting drug by means of delayed elimination. The addition of GELRITE™ (or the like) to a therapeutic formulation of brimonidine tartrate, or derivatives thereof, would make it possible to administer brimonidine tartrate once daily (for example, at night, so as to minimize the impact of mouth dryness, fatigue, drowsiness and eye redness, all possible side effects of the topical administration that could affect compliance).

Another suitable addition to the formulation would be Carbopol 940, a synthetic high molecular weight cross-linked polymer of acrylic acid, which is used to impart a high viscosity to eye drops, thereby increasing their permanence in the conjunctival sac and allowing for a smaller number of daily administrations.

5.5.2 Alternative Modalities of Administration

A possible future development may consist in the use of iontophosresis. This methodology has been used for years clinically to deliver drugs through the skin [Banga et al., 1999; Nair et al., 1999; Pillay et al., 1999] and experimentally to deliver drugs to and through the cornea and the sclera [Friedberg et al., 1991; Church et al., 1992]. Studies have especially focused on iontophoresis as an adjuvant to maximize delivery of drugs against cytomegalovirus, which causes a severe retinal inflammation in patients with AIDS (Lam et al., 1994; Yoshizumi et al., 1996). These studies have shown that iontophoresis is an effective and safe method to deliver drugs that have the retina as a target. Recently, iontophoresis has also been successfully utilized experimentally to induce gene expression in the retina (Asahara et al., 1999). In the future, the use of this method of delivery, which we anticipate may become more and more widespread, may become a suitable approach to maximize the intraocular concentrations attained with the apha-2 adrenergic agonists that are proposed. The delivery of the drug of choice may also be combined with a gene-targeting approach, such as delivering also factor(s) which may enhance the expression of certain genes in retinal cells (custom-designed to disease to be treated). Combination compounds, such as alpha-2 adrenergic agonists and growth factors, may also be delivered through this modality.

Another alternative modality is the use of pluronic gels applied to the scleral surface, taking advantage of the scleral permeability (Lee et al., 1999). This promising approach is in the early stages of development; however, it is premature at this time to predict if and when such a route of “topical” administration will become available.

A third possibility is that of “loading” brimonidine in slow-release ocular inserts to be maintained for several days in the conjunctival sac. This strategy has been proposed for pilocarpine, and anti-glaucoma agent, by Akorn Pharmaceuticals (Buffalo Grove, Ill.) with OCUSERT®. The OCUSERT® system is an elliptically shaped unit designed for continuous release of the drug following placement in the cul-de-sac of the conjunctiva. The OCUSERT® system contains a core reservoir made up of alginic acid. The core is surrounded by a hydrophobic ethylene/vinyl acetate (EVA) copolymer membrane which controls the diffusion of the drug from the OCUSERT® system into the eye. Di(2-ethylhexyl) phthalate is added to increase the rate of diffusion across the EVA membrane. Of the total content of the drug, a portion serves as the thermodynamic diffusional energy source to release the drug and remains in the unit at the end of the week's use. The alginic acid component of the core is not released from the system. The readily visible white margin around the system contains titanium dioxide. The advantage of the OCUSERT® system is the slow release, which occurs over seven days (measured in micrograms per hour). During the first few hours of the seven day time course, the release rate is higher than that prevailing over the remainder of the one-week period. The system releases drug at three times the rated value in the first hours and drops to the rated value in approximately six hours. During the remainder of the 7-day period the release rate is within ±20% of the rated value. A similar system, based on the same general principles, is the OCUFIT SR® [Hubbel et al., 1999]. The only limitation of this system is the limited enthusiasm elicited in Patients may encounter difficulty with the inserts in the application of the inserts and the possible significant foreign-body sensation that the inserts can induce.

5.5.3 Alpha-2 Adrenergic Agonist Compositions

The neuroprotective effect of brimonidine appears to be primarily mediated through stimulation of the alpha-2 adrenergic receptors. Therefore, any alpha-2 agonist should have, at least in part, the same effect observed with brimonidine. Enhanced selectivity and affinity for the alpha-2 receptors will be important to a greater therapeutic effect (or an identical effect at lower doses). A possible class of newly developed drugs may suit the purpose, specifically the imidazolyl-methyl-oxazoles and the imidazolyl-methyl-thiazoles (Boyd et al., 1999).

The initial evidence is that these newly designed compounds have far less sedating effects than the other alpha-2 agonists so far available, which is one of the possible side effects of brimonidine even when administered topically. While the main effect of these new drugs is to provide pain relief, just as much as the other alpha-2 agonists have so far been used only to lower systemic or intraocular pressure, it is expected that imidazolyl-methyl-oxazoles and imidazolyl-methyl-thiazoles will exert at least the same effects observed in vitro and expected in vivo with brimonidine.

### 6.0 REFERENCES

The following literature citations as well as those cited above are incorporated in pertinent part by reference herein for the reasons cited in the above text:

Asahara, T.; Shinomiya, K.; Naito, T.; Shiota, H., “Induction of genes into the rabbit eye by iontophoresis”, Nippon Ganka Gakkai Zasshi, March; 103(3): 178-85, 1999.

Banga, A. K.; Bose, S.; Ghosh, T. K., “Iontophoresis and electroporation: comparisons and contrasts”, Int. J. Pharm., March 1;179(1):1-19, 1999.

Berson et al., “A randomized trial of vitamin A and vitamin E supplementation for retinitis pigmentosa”, Arch. Ophthalmol., 111: 761-772, 1993.

Birnbach, Abstr. #1854, 1994.

Boyd, R. E.; Press, J. B.; Rasmussen, C. R. et al., “Alpha-2 adrenoceptor agonists as potential analgesic agents. 1. (Imidazolylmethyl)oxazoles and -thiazoles”, J. Med. Chem., 42:5064-5071, 1999.

Burke and Schwartz, “Preclinical Evaluation of Brimonidine” Surv. Ophthalmol., 41 Suppl 1:S9-18, 1996.

Cao, Abstr. #1716, 1997.

Carlsson, Chauhan, Lee and Leblanc, “Intraocular Pressure and Progression of Glaucomatous Visual Filed Loss” Am. J. Ophthalmol., 128:697-701, 1999.

Church, A. L.; Barza, M.; Baum, J., “An improved apparatus for transscleral iontophoresis of gentamicin”, Invest Ophthalmol. Vis. Sci., December;33(13):3543-5, 1992.

Cideciyan, Abstr. #1812, 1993.

Eisenfeld, Abstr. #90, 1984.

Erickson, Abstr. #1681, 1987.

Faktorovich, Abstr. #1126, 1990.

Falsini, Abstr. #1881, 1994.

Falsini, Abstr. #1810, 1999.

Farber, Abstr. #677, 1987.

Flannery, Abstr. #1505, 1989.

Friedberg, M. L.; Pleyer, U.; Mondino, B. J., “Device drug delivery to the eye. Collagen shields, iontophoresis, and pumps”, Ophthalmology, May;98(5):725-32, 1991.

Germen, Abstr. #1605, 1997.

Green, Abstr. #1492, 1985.

Green, Abstr. #1830, 1999.

Gu, Abstr. #1446, 1997.

Guérin, Abstr. 1292, 1989.

Guérin, Abstr. #1291, 1993.

Hollyfield, 1974.

Hollyfield, Abstr. #1269, 1979.

Hubbell, H. R.; Noonan, J. S.; Lee, S. B.; Swaminathan, A.; Kaminski, E. A., Edelhauser, H. F., “Scleral permeability of diclofenac released from an Ocufit® SR drug delivery insert”, Invest Ophthalmol. Vis. Sci., 40(suppl 4): S85, 1999.

Jablonski, Abstr. #1783, 1999.

Jablonski, Abstr. #1788, 1999.

Jablonski, Wohabrebbi and Ervin, submitted for publication.

Jacobson, 1967.

Klein, Abstr. #1775, 1999.

Lachkar, Midgal and Dhanjil, “Effect of Brimonidine Tartrate on Ocular Hemodynamic Measurements” Arch. Ophthalmol., 116:1591-1594, 1998.

Lam, T. T.; Fu, J.; Chu, R.; Stojack, K.; Siew, E.; Tso, M. O., “Intravitreal delivery of ganciclovir in rabbits by transscleral iontophoresis”, J. Ocul. Pharmacol., Fall:10(3):571-5, 1994.

Lee, S. B.; Noonan, J. S.; Geroski, D. H.; Prausnitz, M. R.; Edelhauser, H. F., “Drug delivery through the sclera: the influence of thickness, hydration, and sustained release systems”, Invest Ophthalmol. Vis. Sci., 40(suppl 4): S83, 1999.

Lewis, Abstr. #1730, 1992.

Lewis, Abstr. #1710, 1995.

Li, Abstr. #1400, 1991.

Li, Abstr. #1686, 1993.

Li, Abstr. #1874, 1994.

Li, Abstr. #1882, 1995.

Li, Abstr. #1875, 1995.

Li T, Sandberg M A, Pawlyk B S, Rosner B, Hayes K C, Dryja T P, Berson E L. Effect of vitamin A supplementation on rhodopsin mutants threonine-17→methionine and proline -347→serine in transgenic mice and in cell cultures. Proc Natl Acad Sci USA. 1998 September 29;95(20): 11933-8.

Lolley, 1977.

Marlhens, Abstr. #1449, 1997.

Maw, Abstr. #1450, 1997.

Milam, Abstr. #1873, 1998.

Nair, V.; Pillai, O.; Poduri, R.; Panchagnula, R., “Transdennal iontophoresis. Part I: Basic principles and considerations”, Methods Find Exp. Clin. Pharmacol., March;21(2):139-51, 1999.

Nasir, Abstr. #1737, 1997.

Newman, Abstr. #1715, 1996.

Nieuwkoop, 1956.

Okada, Abstr. #1664, 1990.

Osborne, “Inhibition of cAMP Production by Alpha2-Adrenoceptor Stimulation in Rabbit Retina”, Brain Res., 553:84-88, 1991.

Peng et al., 1998.

Pillai, O.; Nair, V.; Poduri, R.; Panchagnula, R., “Transdermal iontophoresis. Part II: Peptide and protein delivery”, Methods Find Exp. Clin. Pharmacol., April;21(3):229-40, 1999.

Redmond, Abstr. #1452,1999.

Reichenbach, Abstr. #1787, 1993.

Reichenbach, Abstr. 1878, 1997

Ripps, Abstr. #1713, 1985.

Rodrigues, Abstr. #1705, 1987.

Roque, Abstr. #1683, 1990.

Santos, Abstr. #1885, 1997.

Seddon, Abstr. #1774, 1998.

Stiemke, 1984.

Steimke, Abstr. #1295, 1994.

Stiemke, 1995.

Wen et al., 1995.

Weng J, Mata N L, Azarian S M, Tzekov R T, Birch D G, Travis G H. Insights into the function of Rim protein in photoreceptors and etiology of Stargardt's disease from the phenotype in abcr knockout mice. Cell. 1999 July 9;98(1):13-23.

Wheeler and Woldemussie, “From the lab to the clinic: Activation of Alpha-2 agonist pathway is neuroprotective in models of Retinal and Optic Nerve Injury” Eur. J. Ophthalmol., 9 Suppl 1:217-21, 1999.

Yoles, Wheeler and Schwartz, “Alpha2-adrenoreceptor Agonists are Neuroprotective in a Rat Model of Optic Nerve Degeneration” Invest. Ophthalmol. Vis. Sci., 40:65078, 1999.

Yoshizumi, M. O.; Roca, J. A.; Lee, D. A.; Lee, G.; Gomez, I., “Ocular iontophoretic supplementation of intravenous foscarnet therapy”, Am. J. Ophthalmol., July;122(l):86-90, 1996.

Zarbin et al, 1986.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the composition, methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims. Accordingly, the exclusive rights sought to be patented are as described in the claims below.

What is claimed is:

1. A method of inhibiting a degenerative condition of a retinal photoreceptor cell, said method comprising contacting a photoreceptor cell having a degenerative condition with a composition comprising a brimonidine compound in an amount effective to inhibit the degenerative condition.

2. The method of claim 1 wherein the brimonidine compound has the following structure: 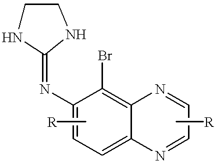
Where R is C₁₋₅ alkyl, Br, Cl or NO₂, and pharmaceutically acceptable salts thereof.

3. The method of claim 1, wherein the brimonidine compound is brimonidine tartrate.

4. The method of claim 1, wherein the amount of brimonidine is between about 0.01% and about 0.05% in a pharmaceutically acceptable vehicle.

5. A method of treating a degenerative condition of retinal photoreceptors, said method comprising administering to a subject in need thereof, a composition comprising a brimonidine compound in an amount effective to delay or reverse said condition.

6. The method of claim 5, wherein the brimonidine compound is administered topically to the eye.

7. The method of claim 5, wherein the amount of brimonidine provides between about 10 and about 1000 nanomolar intraocular concentration.

8. The method of claim 5, wherein said subject is a vertebrate.

9. The method of claim 8, wherein said vertebrate is a mammal.

10. The method of claim 9, wherein said vertebrate is a human being.

11. The method of claim 5, wherein said condition is retinal detachment.

12. The method of claim 5, wherein said condition is age-related macular degeneration.

13. The method of claim 5, wherein said condition is retinitis pigmentosa.

14. A method of reversing or delaying degeneration of a photoreceptor cell in a retina, comprising contacting said retina with a composition that includes an amount of a brimonidine compound effective to inhibit GFAP expression in Müller cells.

15. A method of reversing or delaying degeneration of a photoreceptor cell in a retina, comprising contacting said retina with a composition that includes an amount of a brimonidine compound effective to stimulate upregulation of glutamine synthetase in Müller cells.

16. The method in claim 14 or 15, wherein the brimonidine compound is brimonidine tartrate.

17. The method in claim 14 or 15, wherein the contacting is by topical administration.

18. A kit comprising in suitable container means, a brimonidine composition pharmaceutically suitable for topical administration to the eye, and instructions for administration to a subject in need of treatment for retinal degeneration.

19. A composition comprising a brimonidine compound and at least one human growth factor selected from the group consisting of basic fibroblast growth factor (bFGF), glian-derived neurotrophic factor (CNTF), pigment epithelium-derived factor (PEDF), glial-derived neurotrophic factor (GDNF), and brain-derived neurotrophic factor (BDNF).

20. The composition of claim 19 comprised within a pharmaceutical vehicle suitable for topical administration.

21. A composition comprising a brimonidine compound and a wetting agent.

22. The composition of claim 21 wherein the wetting agent is selected from the group consisting of tyloxapol, polyvinyl alcohol, hydroxyalkyl cellulose, methylcellulose, polyvinyl pyrrolidone, or polyquartemium-10.

23. The composition of claim 19 or 21 further comprising an anti-allergenic/anti-inflammatory drug selected from the group consisting of H1 histamine receptor antagonists, non-steroidal anti-inflammatory compounds (NSAID) and mast cell stabilizers.

24. The composition of claim 23 wherein the brimonidine is brimonidine tartrate, and the anti-allergenic/anti-inflammatory agent is selected from the group consisting of H1 histamine receptor antagonists, Ketotifen hydrochloride, Levocabastine hydrochloride, Olopatadine hydrochloride, emedastine difumarate, Ketorolac tromethamine, Diclofenac sodium, Cromolyn sodium and Lodoxamide tromethamine.
